(19) United States
(12) Patent Application Publication
(10) Pub. No.: US 2022/0039670 A1
(43) Pub. Date: Feb. 10, 2022

(54) HEART FAILURE MONITOR

(71) Applicant: Shifamed Holdings, LLC, Campbell, CA (US)

(72) Inventors: Marwan Berrada, Los Gatos, CA (US); Brian Fahey, Menlo Park, CA (US); Tom Saul, Moss Beach, CA (US)

(21) Appl. No.: 17/413,920

(22) PCT Filed: Dec. 23, 2019
(86) PCT No.: PCT/US2019/068354

§ 371 Date: Jun. 14, 2021

Related U.S. Application Data
(60) Provisional application No. 62/784,036, filed on Dec. 21, 2018.

(51) Int. Cl. A61B 5/0215

(52) U.S. Cl. CPC A61B 5/0215; A61B 2562/028; A61B 2560/0214; A61B 2562/0247

(57) ABSTRACT

Embodiments of device for monitoring pressure in the left atrium are provided. The device is delivered to the left atrium via the coronary sinus. A first portion of the device is deployed in the left atrium, the first portion of the device comprising a pressure sensor. A second portion of the device is deployed in the coronary sinus. Monitoring left atrial pressure via coronary sinus access can provide a safer, less invasive way to monitor a patient for heart failure.

### CROSS-REFERENCE TO RELATED APPLICATION

This application claims the benefit of U.S. Provisional Application No. 62/784,036, filed Dec. 21, 2018, the entire disclosure of which is incorporated by reference herein.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

This application relates to the field of sensing implants. In particular, this application relates to the field of pressure monitoring implants configured for implantation in the heart.

### BACKGROUND

Monitoring implants prove useful in managing a variety of illnesses or for preventing health deteriorations. Such implants allow real-time patient-specific information from patients with special needs and suggest specific behavior (e.g. starting treatment prior to a significant acute event) for patients and physicians. One such case is pressure monitoring in Congestive Heart Failure (CHF) patients, where efforts are made to develop small implants for invasive monitoring of blood pressure changes in the left atrium or other anatomical locations. The purpose is to provide early and accurate detection of a potential heart function decline. Monitoring implants prove useful in treatment of a variety of illnesses or for preventing heath deteriorations by providing prolonged continuous monitoring. Such implants allow real-time patient-specific information for patients with special needs and specific behavior of monitored organs. One such case is pressure monitoring in Congestive Heart Failure (CHF) patients, where efforts are made to develop small implants for monitoring pressure changes in the left atrium or in other anatomical locations, in order to provide early and accurate detection of a potential heart function decline.

In one such example, implants are deployed by advancing a catheter device to the right atrium and performing a transeptal puncture to access the left atrium. In other cases, left atrial implants are surgically implanted in the left atrium through the chest. Both of these methods are associated with risk of medical complications to the patient, sometimes outweighing any provided benefit.

### SUMMARY OF THE DISCLOSURE

In a first aspect, a method of monitoring pressure in a patient is provided. The method comprises advancing a device into a coronary sinus of the patient to a location near the left atrium of the patient; puncturing the wall between the coronary sinus and left atrium to create a passage to the left atrium; and positioning the device, the positioning comprising implanting a first portion of the device comprising a pressure sensor or an array of other MEMS and non-MEMS derived sensors at or near the opening or in the left atrium; and implanting a second portion of the device in the coronary sinus.

The device can comprise a battery, and the positioning can comprise positioning the pressure sensor in the left atrium and positioning the battery in the coronary sinus. In some embodiments, positioning comprising positioning two pressure sensors in the left atrium at two different locations, the method further comprising monitoring pressure at the two locations of the left atrium. In some embodiments, the method comprises using a pressure sensor on the device to monitor blood pressure in the left atrium. The method can comprise monitoring pressure at one or a plurality of locations in the left atrium. In some embodiments, the method comprises monitoring pressure in the left atrium and the coronary sinus. Positioning the first portion of the device can comprise positioning the first portion at or near the puncture site. In some embodiments, positioning the first portion of the device comprises positioning the first portion in the left atrium. Positioning the first portion of the device can comprise anchoring the first portion at or near the puncture site. In some embodiments, the anchoring comprises deploying a tether within the left atrium. The anchoring can comprise deploying a nitinol anchor on at least one side of the puncture site. In some embodiments, the anchoring comprises deploying a first self-expandable anchor on the left atrium side of the opening and a second self-expandable anchor on the coronary sinus side of the puncture site, the first and second anchors configured to grasp the wall between the coronary sinus and left atrium. Positioning a second portion of the device can comprise anchoring the second portion within the coronary sinus. In some embodiments, the method comprises positioning a second pressure sensor in the left atrium. The method can comprise positioning a third portion at a location remote from the coronary sinus, wherein the third portion can be one of a pacemaker pocket, diaphragm, and/or endovascular power supply. In some embodiments, the method comprises implanting a power source comprising a can at a subcutaneous, endovascular, and/or subxiphoid location remote to the device, and connecting the can or power source to the pressure sensor. The method can comprise substantially sealing the opening with a portion of the device.

In another aspect, a device for monitoring blood pressure in the left atrium is provided. The device comprises a pressure sensor configured to be implanted at or near a puncture site between a left atrium and coronary sinus of a patient; and control electronics configured to be positioned remote from and connected to the pressure sensor.

In some embodiments, at least part of the control electronics is configured to be positioned in the coronary sinus. The part of the control electronics can be hard-wired to the pressure sensor. In some embodiments, the part of the control electronics and the pressure sensor are wirelessly-connected. The control electronics can comprise a battery, memory, and a controller for controlling the pressure sensor. In some embodiments, the device comprises an antenna unit. The control electronics can comprise an antenna unit. The antenna unit can be remote from the pressure sensor. In some embodiments, the antenna unit is remote from the pressure sensor and the control electronics. In some embodiments, the device comprises a power source that is remote from the control electronics.

In another aspect, a device for monitoring pressure in the left atrium is provided. The device comprises a first anchor configured to be positioned in the left atrium; a second anchor configured to be positioned in the coronary sinus; and a body extending between the first and second anchors and configured to be positioned such that at least a portion of the body that is between the first and second anchors is configured to be positioned in between the left atrium and the coronary sinus, the body comprising a pressure sensor and an antenna unit.

In some embodiments, the pressure sensor is positioned between the first and second anchors. A first end of the body portion can extend beyond the first anchor. In some embodiments, the pressure sensor is positioned between the first end and the first anchor on the body portion. At least one of the first and second anchors can comprise a plurality of legs. In some embodiments, the first anchor and the second anchor are axially translatable relative to one another along a longitudinal axis of the device extending between the first and second anchor. In some embodiments, the first anchor and the second anchor are translatable relative to one another in a direction substantially perpendicular to a longitudinal axis of the device extending between the first and second anchor. At least one of the first anchor and the second anchor can be configured to pivot with respect to the body portion. In some embodiments, the body is configured to be positioned within and substantially seal an opening created between the coronary sinus and the left atrium.

In yet another aspect, a method of monitoring pressure in a patient is provided. The method comprises advancing a first device and a second device through a coronary sinus of the patient to a location near the left atrium of the patient; puncturing the wall between the coronary sinus and left atrium to create an opening to the left atrium; positioning the first device comprising a pressure sensor at or near the opening or in the left atrium; and positioning a second device in the coronary sinus.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1A-1B show embodiments of pressure monitoring devices.

FIG. 2 illustrates another embodiment of a pressure monitoring device.

FIGS. 3A-3B depict translation of device components permitted by embodiments of a pressure monitoring device.

FIGS. 4A-4D show an embodiment of a method of implanting a pressure monitoring device.

FIG. 5A shows an embodiment of a delivery tool for delivering the implant of the current application.

FIG. 5B illustrates various entries and deployment routes for advancing to the implantation site.

FIG. 6 depicts an embodiment of a delivery catheter advanced through the coronary sinus to the left atrium.

FIG. 7 shows an embodiment of a device implanted with a portion in the coronary sinus and a portion in the left atrium.

### DETAILED DESCRIPTION

Provided herein are devices configured to monitor pressure in the left atrium. The devices can be adapted to access the left atrium from the coronary sinus, providing a safer deployment procedure than other left atrial implants. The device can comprise a first portion configured to be implanted in the coronary sinus, and another portion configured to be implanted in the left atrium. One or more pressure sensors on the device are configured to monitor at least blood pressure in the left atrium.

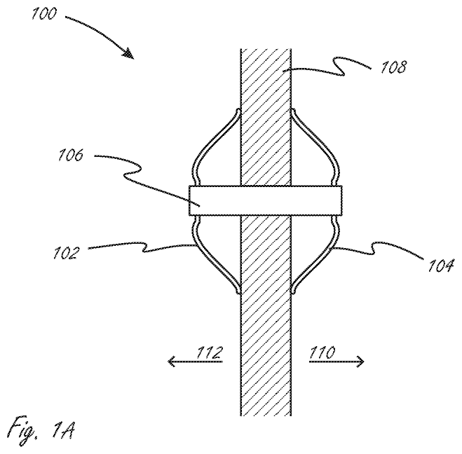

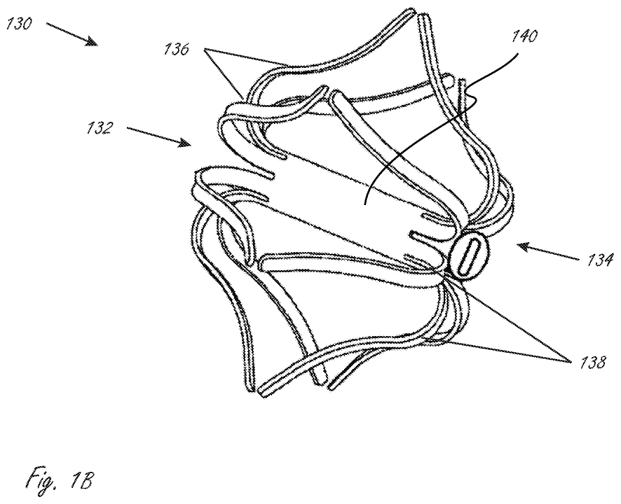

FIG. 1A depicts an embodiment of a device 100 comprising a first anchor 102, a second anchor 104, and a body portion 106 extending between the first anchor 102 and the second anchor 104. The first anchor 102 is configured to be implanted in the left atrium 112. The second anchor 104 is configured to be implanted in the coronary sinus 110. The body portion 106 is configured to be positioned at least partially within the wall 108 between the coronary sinus and the left atrium. The first and/or second anchors can comprise a plurality of legs and be configured to self-expand upon release from a constrained or biased position (e.g., within a delivery sheath). In some embodiments, the first and/or second anchors can comprise a conical or disc-shaped mesh that is configured to self-expand upon release from a constrained or biased position. One or more pressure sensors or other MEMS and non-MEMS based sensors can be positioned on the device, for example, along the body portion of the device, as described in further detail below. In some embodiments, the pressure sensor(s) can be positioned on the first anchor of the device.

In some embodiments, portions or all of the device can comprise a self-expanding material such as nitinol. In alternative embodiments, portions of the device can comprise a malleable material such as aluminum, copper, or annealed stainless steel that is mechanically adjusted to be in a desired configuration as part of the deployment process.

FIG. 1B illustrates another embodiment of a device 130 configured to be positioned between the coronary sinus and the left atrium. The device 130 comprises a first or distal anchor 132 and a second or proximal anchor 134 connected by a central body portion 140. The distal anchor 132 includes a plurality of legs 136 which originate at a distal end of body portion 140. In some such embodiments, each leg comprises a free leg end (i.e., an end that is not connected to another portion of the device or another structure). As described above with respect to device 100, distal anchor is self-expandable from a fully closed or substantially-fully closed formation, in which legs 136 are substantially straighten and extend axially to body 140 and distally, to a predetermined non-stressed shape, in which legs 136 extend laterally to body 140 and proximally. The proximal anchor is self-expandable from a fully closed or substantially-fully closed formation, in which legs 138 are substantially straight to a predetermined non-stressed shape, in which legs expand radially. In some embodiments, the device 130 is designed for optional withdrawing from an original implantation site in a wall target and relocating to another, for example in order to improve safety of efficacy of the implant by choosing a more appropriate anatomical location, optionally patient-specific, as the final implantation site, optionally after at least one previous attempt. In some such embodiments, legs 136 and/or leg 138 are designed and configured for recollapsing into a delivery device. As shown in FIG. 1B, at the non-stressed shape, legs 136 and legs 138 in distal anchor 132 take a form similar to “spider's legs” which provides improved elasticity, especially in longitudinal axis direction, as well as capability to recollapse into a tubular by realigning and straightening distally.

The distal anchor can be configured to be positioned within the left atrium. The proximal anchor can be configured to be positioned within the coronary sinus. A pressure sensor can be positioned along a length of the body portion. In some embodiments, a pressure sensor is inserted within the body portion. In some embodiments, the pressure sensor can be positioned on one or more of the device legs. Any one or a combination of the described locations for a pressure sensor can be utilized in a device embodiment. Further details about device 130 are provided in U.S. Patent Publication No. 20150157268, the entire disclosure of which is hereby incorporate by reference herein.

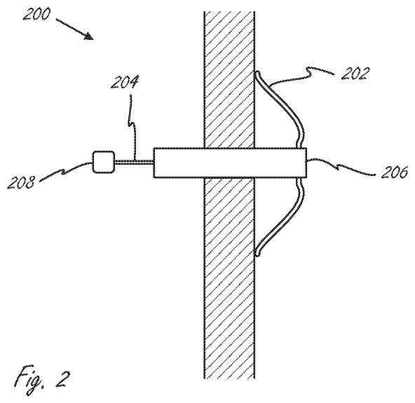

Other configurations for the device are also possible. For example, FIG. 2 shows an embodiment of a device 200 comprising an anchor 202 connected to a tether 204 by a central body portion 206. The tether can comprise a pressure sensor 208 attached along its length, for example at its distal end. In some embodiments, multiple pressure sensors can be positioned along the tether. In some embodiments, multiple tethers comprising multiple pressure sensors can be used.

As previously described, the device comprises one or more pressure sensors (e.g., a pressure transducer comprising a membrane or surface sensitive to pressure changes). The device can also comprise an entire measurement unit comprising at least one of a capacitor (for example, in the case of a capacitive-based MEMS transducer), at least one electrical component (for example, any of a telemetry unit, a motherboard, a memory, a rechargeable and non-rechargeable battery, an amplifier, an antenna, a sensor, or other), a microprocessor, FPGA, an application-specific integrated circuit (ASIC), adapted to convert a reading such as the MEMS capacitance to a frequency-encoded or otherwise desirable signal, a transmitter and/or antenna designed to transmit (encrypted, non-encrypted, compressed, non-compressed) data to a remote receiver (not shown) provided outside patient's body or subcutaneous to the body, allowing a wireless connection for transmitting sensed data either in real-time or as a packet of pre-recorded information, and, optionally, means for collecting remote power such as a power receiver configured for receiving powering energy transmitted wirelessly from a remote source. In some embodiments, the support electronics are provided within the body portion of the second anchor configured to be implanted in the coronary sinus. It can be advantageous to limit structure in the left atrium to mitigate the risk of thromboembolic events. In some embodiments, support electronics can be carried on a portion configured to be anchored or floated outside of the coronary sinus, such as within the right atrium. The communications to the support electronics can be wired or wireless. Such embodiments can help limit occlusion and possible vessel stresses to the coronary sinus.

In some embodiments, the antenna can be remote to the pressure sensor. For example, in some embodiments, the anchoring structure of the device can function as both the anchor and as the antenna. An anchor that comprises sufficient material and a properly configured shape (e.g., multiple coils of a metallic material) can function as an antenna. The antenna can be remote from the pressure sensor, and yet within the body blood vessels. In some embodiments, the antenna unit is located in the epicardial space, but tethered to the device through the myocardium. In some embodiments, the antenna is remote from the first and second portions of the device. For example, the antenna can be positioned on the body portion of the device. In some embodiments, the body portion of the device can serve as the antenna. For example, a body portion comprising sufficient material and a properly configured shape (e.g., multiple coils of a metallic material) can function as an antenna. In some embodiments, the system can make use of other materials and components for communication. For example, the system may connect with existing implantable devices (e.g. pacemakers, ICDs, ventricular assist devices, cardiac monitors, etc.) to make use of their processing power, communication systems, and/or memory.

In the devices described above, a central body portion connects a first and second portion of the device. The body portion can comprise various configurations. For example, in some embodiments, the body portion is solid, for example, comprising a solid generally cylindrical shape. The pressure sensor can be positioned along the solid body portion. In some embodiments, the body portion can be open, creating a tubular portion. In certain such embodiments, the tubular portion can be configured to receive a pressure sensing portion of the device. In other such embodiments, the pressure sensor is positioned on the open body portion. Other components (e.g., power source, antenna, other sensor, a plug etc.) can be inserted into the open body portion. The portion inserted into the body of the device can be configured to fully seal the open body portion. In some embodiments, the body portion is sized and shaped such that any opening created by puncture of the wall between the left atrium and the coronary sinus is filled and sealed by the body portion. In some embodiments, the body portion is positioned within and does not extend part the first and second portions. In other embodiments, the body portion extends past at least one of the first and second portions. The pressure sensor(s) can be positioned on a portion of the body that extends past the first or second portions or a portion positioned between the first and second portions. In some embodiments, the pressure sensor comprises a sensing element positioned in the coronary sinus fluidically coupled to a deformable element in the atrium. In such embodiments, the term ‘pressure sensor’ can refer to the sensing element portion of the sensor.

As noted previously, in some embodiments, the device comprises multiple pressure sensors configured to monitor pressure at multiple, different locations in the left atrium. Multiple pressure readings can provide a more complete and accurate picture of the physiological state of the heart.

In some embodiments, the distal aspect of the device may be configured such that in a first configuration, a beveled edge or otherwise sharp tip may be exposed. In such an embodiment, the device itself could be utilized as the puncturing mechanism to create the passageway between the coronary sinus and the left atrium, obviating the need for separate puncture tools or separate puncture steps to create a hole in the left atrial wall. In a method of use, the sharp tip is initially recessed or otherwise covered such that the leading edge of the device presents an atraumatic distal end while the delivery mechanism navigates it into the coronary sinus. Once in the proper position within the coronary sinus, the sharpened tip is exposed, for example by partially withdrawing a protective sheath or by advancing a tip from an initial position inside of a catheter lumen into a second position beyond the distal boundary of the catheter. The sharpened tip may be utilized to create a passageway into the left atrium, which simultaneously advances the distal portion of the device into the left atrium. Once within the left atrium, an action such as the withdrawal of a sheath may deploy a device feature such as an anchor or a tether, at which point the sharpened tip is covered or otherwise obscured such that there is no risk of additional unwanted tissue injury.

In some embodiments, multiple devices can be used to monitor pressure in the left atrium via coronary sinus access. For example, a first device comprising a pressure sensor can be positioned in the left atrium. A second device can be positioned in the coronary sinus. In some embodiments, multiple pressure sensors are recording data simultaneously. In some embodiments, the pressure differential between sensors in the left atrium and coronary sinus is utilized to adjust a parameter. Multiple sensors or devices can advantageously allow for the collection of additional data, providing increased accuracy and allowing measurement of drift.

In some embodiments, the pressure sensor(s) of the device can be configured to be embedded within the wall between the left atrium and coronary sinus. In some embodiments the pressure sensor can extend into the left atrium a distance of about 2.5 cm (e.g., about 1-4 cm, about 2-3 cm, etc.).

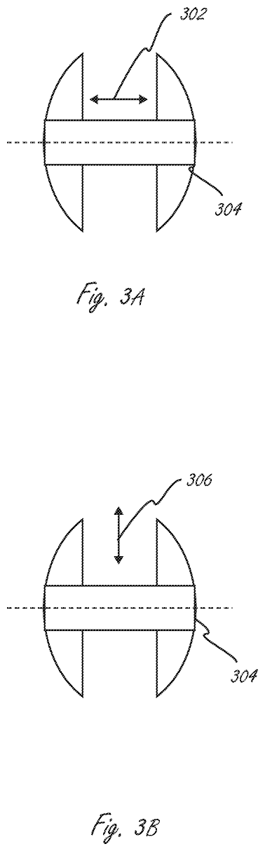

A concern with devices implanted in walls around the heart is fatigue and fracturing. To address these concerns, in some embodiments, the device comprises multiple pivot points to allow the device to move and flex along with the heart wall. For example, the device can comprise one or more pivot points when an anchor attaches to the body portion. The device can comprise one or more pivot points along the anchor. In some embodiments, as shown in FIGS. 3A and 3B, the device comprises sliders that allow the first and second portions of the device (e.g., first and second anchors) to slide relative to one another along a longitudinal axis 304 running through the body portion of the device as indicated by arrows 302. For example, the sliders can comprise telescoping portions of the device that allow the length of a portion of the device (e.g., the body portion) to increase and/or decrease based on stresses provided by the heart wall. In some embodiments, the device comprises sliders that allow first and second portions of the device (e.g., first and second anchors) to move relative to one another in a direction substantially perpendicular to a longitudinal axis 304 of the device extending between the first and second anchor, indicated by arrows 306. Allowing the components of the device to move relative to one another can reduce stress on components of the device and prolong its life. Other configurations are also possible. For example, the body portion can be configured to stretch and contract to allow flexing of the wall between the first and second portions.

In some embodiments, the device power source can be positioned remote to the device. For example, the power source can be placed subcutaneously similar to a pacemaker generator. The power source (and related electronics) can be configured for implantation in a pacemaker pocket. The power source can be placed at a subxiphoid location, in some embodiments. For another example, the power source can be placed in a remote endovascular location. A benefit of this configuration is that the power source can be placed percutaneously, without requiring opening of tissue during surgery. In some embodiments, the power supply can be configured to wirelessly transfer power to the one or more pressure sensors. For example, the remote power source or can may comprise an antenna adapted to recharge the sensor or query the sensor, in the case of a passive sensor. Other configurations (e.g., on board power source, inductively charged power source) are also possible. In some embodiments, the device comprises a local power source (e.g., a battery, capacitor, etc.). The local power source can be combined with an energy harvesting mechanism (e.g., using RF, magnetic, acoustic, motion, etc.)

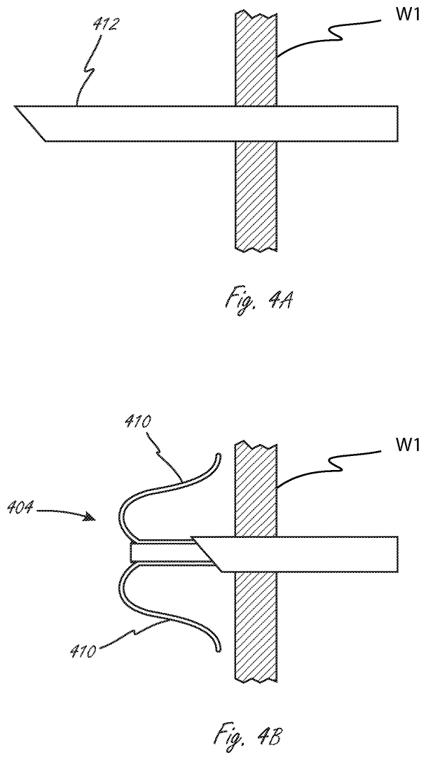

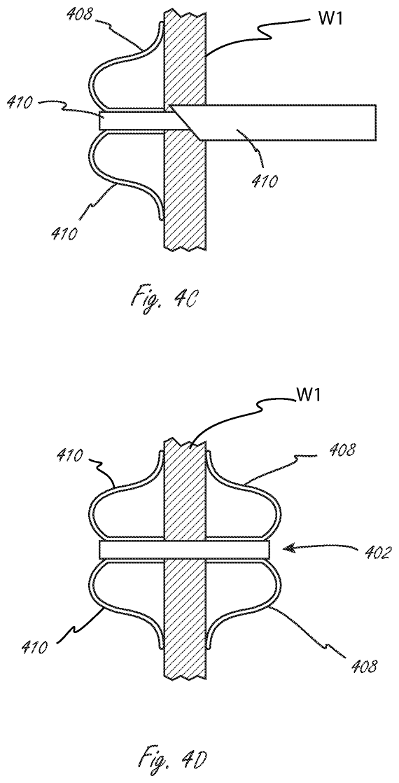

FIGS. 4A-4F schematically illustrate delivery, implantation, and deployment of the device (e.g., like device 130) of the current application. As described above, the device comprises a proximal end, a distal end, and a body connecting the proximal and distal ends. The body comprises a pressure sensor disposed thereon. A flexible proximal anchor 402 coupled to the body 406 comprising a plurality of legs 408, each projection having a free end, and a flexible distal anchor 404 coupled to the elongate body 406 comprising a plurality of legs 410, each leg having a free end. FIG. 4A illustrates delivery of the implant 400 via a delivery device, such as sheath 412, into a wall W1 between the left atrium and coronary sinus via a coronary sinus approach. The coronary sinus can be entered at its outflow in the right atrium. FIG. 4B illustrates deploying the flexible distal anchor from the delivery device 412 so that it self-expands from a collapsed configuration, in which the plurality of legs 410 are substantially straight and extend distally of the body 406, to a non-stressed expanded configuration in which the plurality of legs 410 extend laterally to the body 406 and proximally. FIG. 4C shows an optional verification step which may be performed by the medical practitioner and includes pulling of device proximally (backward) to confirm correct anchoring to wall W1 and deployment of legs 410.

FIG. 4D illustrates the device after deploying the flexible proximal anchor from the delivery device so that it self-expands from a collapsed configuration in which the plurality of legs 408 are substantially straight and extend proximally to an expanded configuration in which the plurality of legs 408 expand radially. The legs 408 effectively shorten and spring towards the body 406 in the expanded configuration so as form symmetrical proximal and distal anchors having spider leg shapes that mirror each other for increased flexibility on both the inner and outer surfaces of wall W1, which is of particular advantage in highly pulsatile/muscular heart wall structures. In particular, the free ends of the proximal and/or distal anchors can be configured to engage the surface of the coronary sinus wall and the left atrial wall, respectively. In some embodiments, the flexible proximal and distal anchors are formed from a single anchor assembly. In such a configuration, the plurality of legs 408 originate at a proximal end of the body, each leg ending with a free end and the plurality of legs 410 originate at a distal end of the body, each leg ending with a leg free end. It will be appreciated that the proximal and distal anchors may also be formed from separate assemblies.

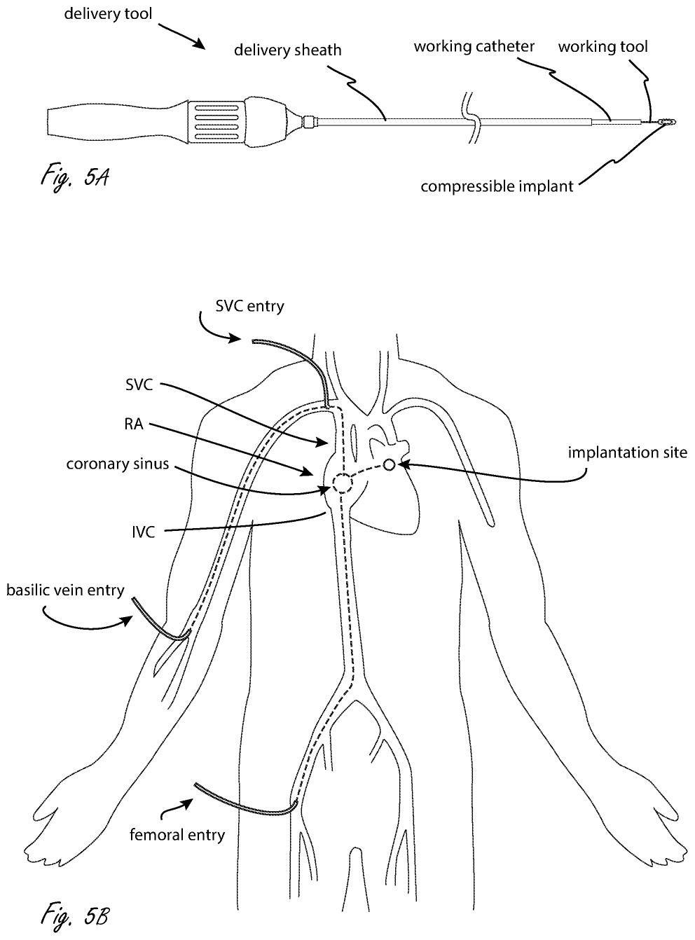

FIG. 5A illustrates an embodiment of a delivery tool that can be used for delivering the implants of the current application. The tool can comprise a handle which can comprise one or more controls. The handle is connected to a delivery sheath. A working catheter extends through and within the delivery sheath. A working tool can be positioned within the working tool. As shown in FIG. 5A, the implant can be connected to the working tool. The implant can be exposed from within the sheath during deployment to cause self-expansion.

FIG. 5B illustrates various entries and deployment routes for implanting the devices described herein. In some embodiments, the femoral vein serves as the entry site. Through that site, the delivery sheath and implant can be advanced to the coronary sinus, and ultimately to the implant site. In some embodiments, the basilic vein can be used as the entry site. Similarly, the delivery sheath and implant are advanced from there to the coronary sinus, and then to the implant site. In some embodiments, the superior vena cava can be used for entry. From there, the delivery sheath and implant can be advanced to the coronary sinus and implant site.

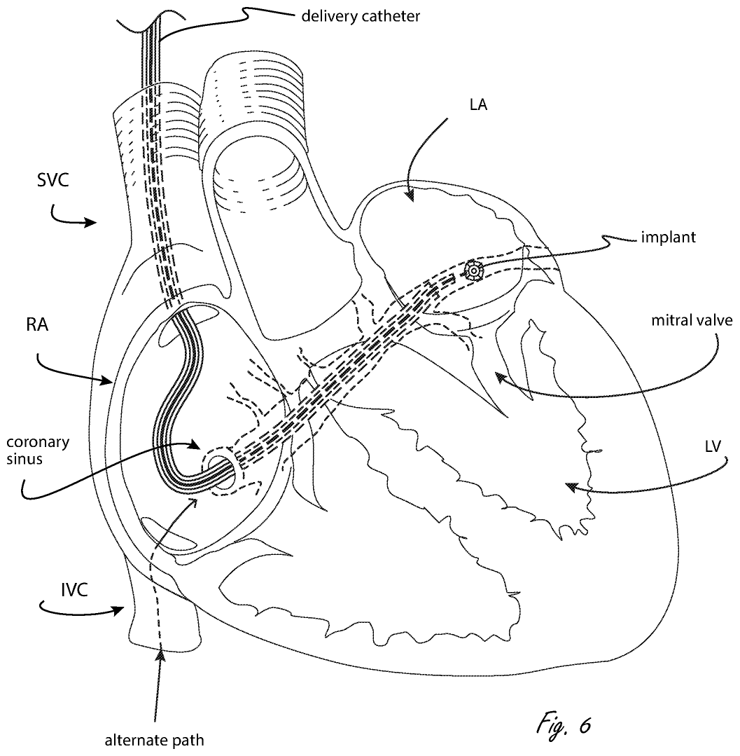

FIG. 6 shows an embodiment of implant deployment in which the superior vena cava is used as the entry site. The delivery sheath and implant are advanced into the right atrium. From the right atrium, the sheath and implant are navigated through the coronary sinus to the left atrium. The sheath and implant are advanced through a puncture between the coronary sinus and left atrium. FIG. 6 shows the device after deployment from the delivery sheath. A portion of the device (e.g., anchor) in the coronary sinus is visible in FIG. 6.

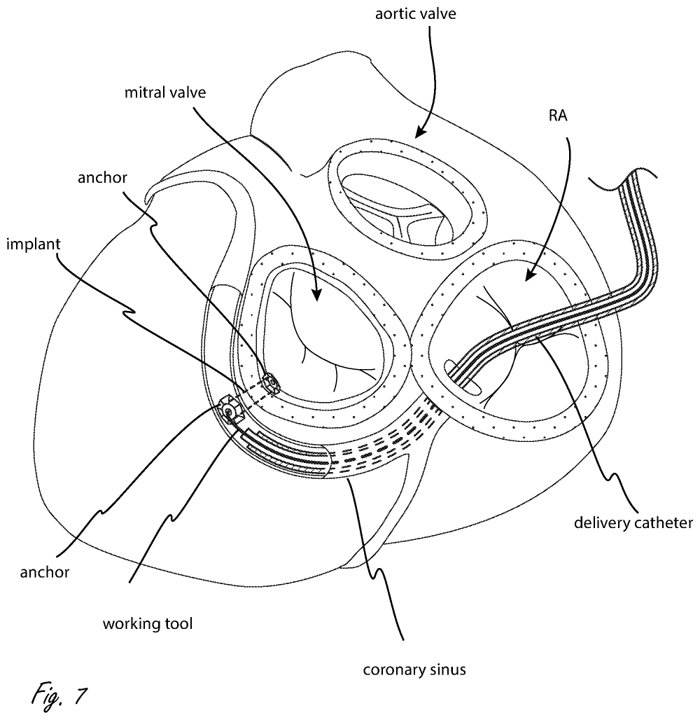

FIG. 7 illustrates a close up view of the implant deployment. The delivery sheath is advanced through right atrium to the coronary sinus, and ultimately to the right atrium. The sheath and implant can be advanced into the left atrium. The sheath can be withdrawn to expose a portion of the implant (e.g., anchor and pressure sensor) in the left atrium, causing that portion to self-expand. The sheath and implant are then withdrawn through the puncture site, back into the coronary sinus. There, the sheath can be withdrawn to expose another portion of the implant (e.g., anchor), causing that portion to self-expand, thereby implanting the device. In some embodiments, the procedure can be performed under fluoroscopy to ensure proper positioning prior to release of the implant.

In some embodiments, portions or all of the device can be configured for tissue ingrowth. In some embodiments, tissue may grow over a bare material of the device (e.g., metal). In some embodiments, the anchor is configured to promote tissue ingrowth and/or healing. In some embodiments, a tissue ingrowth matrix or agent may be provided on portions of the device. In some embodiments, the anchor comprises polyester felt.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present.

Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A method of monitoring pressure in a patient, the method comprising
advancing a device into a coronary sinus of the patient to a location near the left atrium of the patient;
puncturing the wall between the coronary sinus and left atrium to create an opening to the left atrium; and
positioning the device, the positioning comprising
implanting a first portion of the device comprising a pressure sensor at or near the opening or in the left atrium; and
implanting a second portion of the device in the coronary sinus.

2. The method of claim 1, further comprising using a pressure sensor on the device to monitor pressure in the left atrium.

3. The method of claim 1, wherein the device includes a battery, wherein the positioning comprises positioning the pressure sensor in the left atrium and positioning the battery in the coronary sinus.

4. The method of claim 1, wherein the positioning comprises positioning two pressure sensors in the left atrium at two different locations, the method further comprising monitoring pressure at the two locations of the left atrium.

5. The method of claim 1, further comprising monitoring pressure in the left atrium and the coronary sinus.

6. The method of claim 1, wherein positioning the first portion of the device comprises positioning the first portion in the opening.

7. The method of claim 1, wherein positioning the first portion of the device comprises positioning the first portion in the left atrium.

8. The method of claim 1, wherein positioning the first portion of the device comprises anchoring the first portion at the opening.

9. The method of claim 8, wherein the anchoring comprises deploying a tether within the left atrium.

10. The method of claim 8, wherein the anchoring comprises deploying a nitinol anchor on at least one side of the opening.

11. The method of claim 8, wherein the anchoring comprises deploying a first self-expandable anchor on the left atrium side of the opening and a second self-expandable anchor on the coronary sinus side of the opening, the first and second anchors configured to grasp the wall between the coronary sinus and left atrium.

12. The method of claim 1, wherein positioning a second portion of the device comprises anchoring the second portion within the coronary sinus.

13. The method of claim 1, further comprising positioning a second pressure sensor in the left atrium.

14. The method of claim 1, further comprising positioning a third portion at a location remote from the coronary sinus, wherein the location is one of a pacemaker pocket, diaphragm, and/or endovascular power supply.

15. The method of claim 1, further comprising implanting a power source comprising a can at a subcutaneous, endovascular, and/or subxiphoid location remote to the device, and connecting the can or power source to the pressure sensor.

16. The method of claim 1, further comprising substantially sealing the opening with a portion of the device.

17-33. (canceled)

34. A method of monitoring pressure in a patient, the method comprising
advancing a first device and a second device through a coronary sinus of the patient to a location near the left atrium of the patient;
puncturing the wall between the coronary sinus and left atrium to create an opening to the left atrium;
positioning the first device comprising a pressure sensor at or near the opening or in the left atrium; and
positioning the second device in the coronary sinus.
